(19) United States
(12) Patent Application Publication
(10) Pub. No.: US 2014/0124557 A1
(43) Pub. Date: May 8, 2014

(54) Knee Brace Holster

(71) Applicant: David M. Velarde, Knoxville, TN (US)

(72) Inventor: David M. Velarde, Knoxville, TN (US)

(21) Appl. No.: 14/150,433

(22) Filed: Jan. 8, 2014

Related U.S. Application Data
(63) Continuation-in-part of application No. 13/785,404, filed on Mar. 5, 2013.
Application No. 13/785,404 is a continuation-in-part of application No. 13/603,566, filed on Sep. 5, 2012.
(60) Provisional application No. 61/606,704, filed on Mar. 5, 2012.
(60) Provisional application No. 61/638,870, filed on Apr. 26, 2012.

(51) Int. Cl. A45F 5/00; A61F 5/01

(52) U.S. Cl. CPC A45F 5/00; A61F 5/0123
USPC 224/576

(57) ABSTRACT

A knee brace holster is disclosed herein. A knee brace is provided which is configured to wrap around at least a portion of a leg of a user proximate a knee of the user to provide support to the user's knee. A mount is provided for mounting a holster to an outer surface of said knee brace.

### CROSS-REFERENCE TO RELATED APPLICATIONS

This application is a continuation-in-part of U.S. patent application Ser. No. 13/785,404, filed Mar. 5, 2013, which is a continuation-in-part of U.S. patent application Ser. No. 13/603,566, filed Sep. 5, 2012, and which claims the benefit of U.S. Provisional Patent Application No. 61/606,704, filed on Mar. 5, 2012, and U.S. Provisional Patent Application No. 61/638,870, filed on Apr. 26, 2012, each of which is incorporated in its entirety herein by reference.

### STATEMENT REGARDING FEDERALLY-SPONSORED RESEARCH OR DEVELOPMENT

Not Applicable

### BACKGROUND OF THE INVENTION

1. Field of Invention

The present general inventive concept relates to a knee holster for securing an object, such as for example a firearm or other object, to a knee of a wearer, and more particularly, to a knee holster having apparatus for providing bracing support to the knee and for limiting movement of the holster in relation to the knee.

2. Description of the Related Art

Numerous designs of carrying devices for securing tools and other articles to a body of a person are known in the art. For example, in the field of firearms and firearm accessories, it is known to provide a holster or other similar carrying device to secure a firearm or other accessory to the body of a user, for example to facilitate hands-free carrying of the firearm or other accessory. The term “holster” is often used to refer specifically to a carrying device for securing a firearm, and in particular a handgun, to the body of a user. However, as used herein, the term “holster” will be understood to include a carrying device for securing any of a number of articles, such as for example firearms, ammunition, ammunition magazines, flashlights, knives, hand tools, navigation devices, communication devices, aiming devices, personal protection devices, non-lethal weapons, handcuffs and other restraint devices, personal documents, etc. For convenience, the term “article” as used herein will be understood to include any of a number of articles, including but not limited to the above-discussed articles, which a user may desire to secure to the user's body.

Numerous types of holsters for securing articles, and in particular firearms, to an outer surface of a person's leg between the knee and waist are known in the art. Such holsters are usually designed to hang via supporting straps from a belt worn around the person's waist, and may also include one or more additional straps or belts to wrap around the person's thigh. The straps supporting these holsters are typically adjusted to be substantially snug and/or taught when the person stands upright, thereby securing the holster firmly against the outer surface of the person's leg. However, in some instances, such straps can limit bending movement of the person's leg near the hip or waist. In other instances, if the wearer of such a holster bends near the waist or hip, the supporting straps of the holster may temporarily loosen or become slack. In such instances, the holster may temporarily become unstable in relation to the person's leg, such that the holster may shift, swing, or otherwise move in relation to the person's leg.

Firearms are relatively heavy items, and when the above-described holsters are allowed to loosen while containing a firearm, they have a tendency to shift around during the articulated movement of the leg, especially when the wearer is engaging in intense physical activity such as running, kicking, crawling, etc. Such shifting can result in impact to the wearer's leg, as well as compromised support of the firearm, impairing smooth articulated movement of the wearer's leg and leading to discomfort and/or injury to the wearer. Such shifting can also result in decreased accessibility of the item carried in the holster by the user. In the case of a firearm secured in a holster, it is important for fast and precise access and drawing of the firearm that the holster remain at a fixed position in relation to the leg, so that when a hand reaches down to withdraw the firearm, the firearm is in the fixed position in relation to the leg as expected by the wearer. In the case of so-called “retention holsters,” in which the holster is made to fit a firearm snugly to establish a releasable frictional connection between the firearm and the holster, shifting of the holster along the length of the wearer's leg can impede, and in some instances even prevent, drawing of the firearm from the holster.

To discourage shifting of a holster and associated firearm in relation to the wearer's leg, it is customary to secure the belts or straps of the holster very tightly around the leg, often times so tightly that vascular circulation to the wearer's leg is constricted or restricted. Such tight securement of the holster can result in discomfort to the user, such as for example by allowing the firearm and/or holster to dig into the skin and flesh in the wearer's leg. Such discomfort can be significant, and can even result in physical injury, and is especially likely when the holster is worn for extended periods of time or through intense physical activity. Furthermore, it is uncommon for a holster designed for securing an article to an outer surface of a wearer's leg to also provide support to the knee of the user during normal movement of the wearer's leg.

In light of the above, there is a need in the art for a holster which allows securement of a firearm or other article to an outer portion of a leg of a wearer and which provides support to the knee of the user while limiting movement of the holster in relation to the wearer's leg. Furthermore, there is a need for a holster which limits movement of the holster along the length of the wearer's leg during removal of an article from the holster, but which also allows for increased comfort to the wearer of the holster.

### BRIEF DESCRIPTION OF THE SEVERAL VIEWS OF THE DRAWINGS

The above-mentioned features of the invention will become more clearly understood from the following detailed description of the invention read together with the drawings in which:

FIG. 1 is a perspective view showing one embodiment of a knee brace holster constructed in accordance with several features of the present general inventive concept;

FIG. 2 is a partially exploded perspective view showing the knee brace holster of FIG. 1;

FIG. 3 is an exploded perspective view of a portion of the knee brace holster of FIG. 1;

FIG. 4 is a close-up, partially exploded perspective view of the rotatable connector portion of the knee brace holster of FIG. 1;

FIG. 5 is an exploded perspective view of the holster portion of the knee brace holster of FIG. 1;

FIG. 6 is a side elevation view of the knee brace holster of FIG. 1 worn on a leg of a wearer, with a holster and associated firearm mounted thereon;

FIG. 7 is a side elevation view of another embodiment of a knee brace holster worn on a leg of a wearer, with a pair of holsters and associated firearm magazine and handcuffs mounted thereon;

FIG. 8 is an exploded perspective view of one embodiment of a holster for use in carrying a cellular device; and

FIG. 9 is an exploded perspective view of another embodiment of a holster for use in carrying personal documents.

### DESCRIPTION OF THE INVENTION

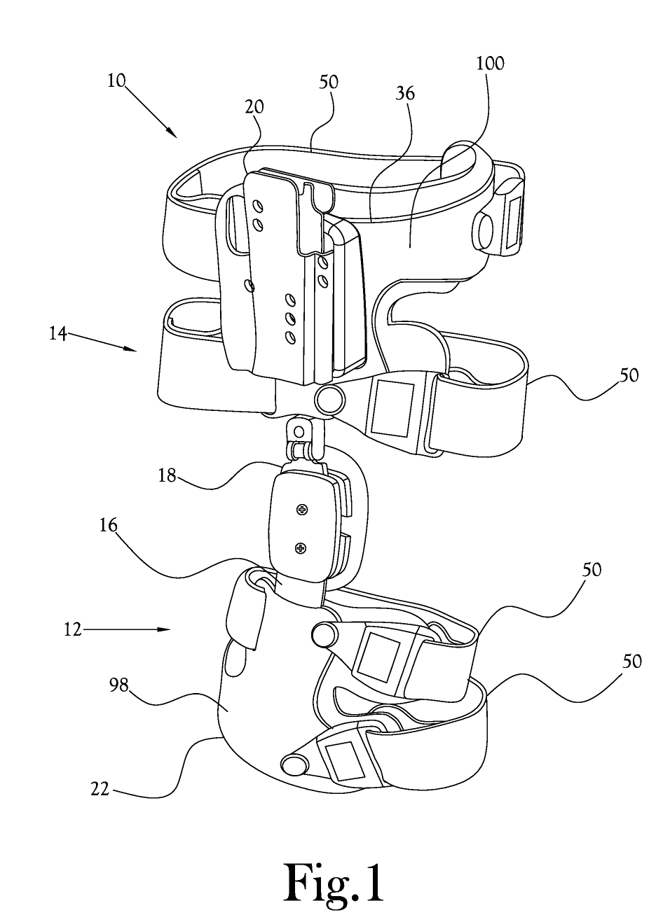

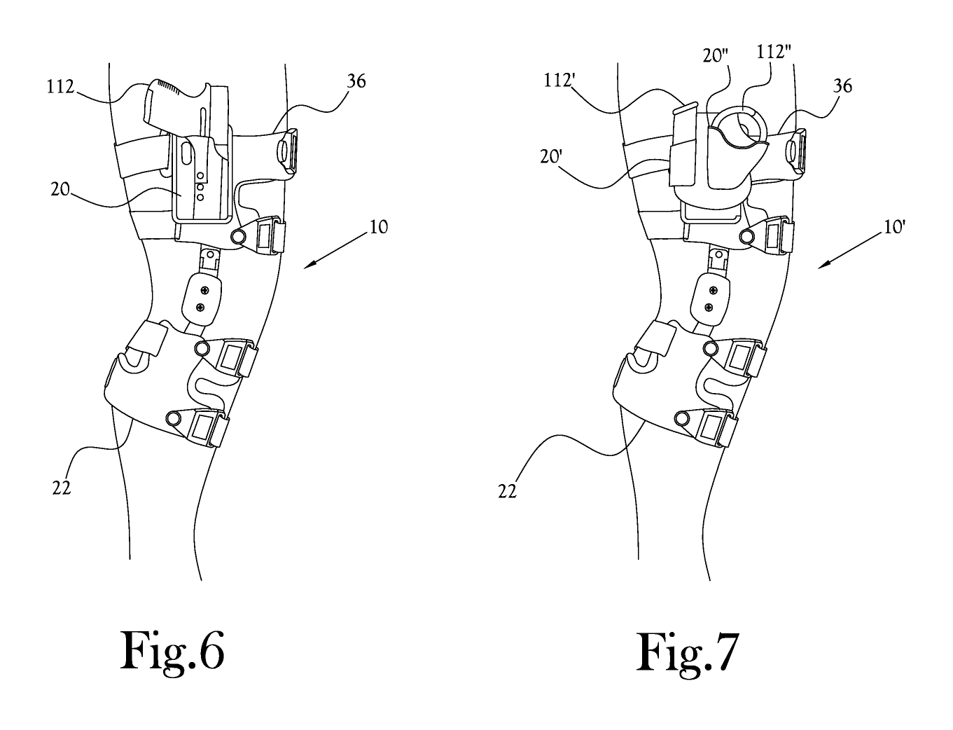

In accordance with several features of the present general inventive concept, a knee brace holster for securing an article to a leg of a wearer while also providing support to the knee of the wearer and limiting movement of the holster in relation to the wearer's leg is disclosed herein. Referring to FIG. 1, in one embodiment of the present general inventive concept, a knee brace holster 10 is provided that includes generally a lower leg portion 12 movably, and preferably rotatably, connected to an upper leg portion 14 via connecting members 16, 18. As will be further discussed below, in several embodiments, at least one holster 20 is secured along an exterior surface of one of the leg portions 12, 14 by suitable fasteners, such that when upper and lower portions of a wearer's leg are disposed within the upper leg portion 14 and lower leg portion 12, respectively, an article 112 (see FIG. 6) may be retained within the holster 20 and thereby held against the wearer's leg for convenient access thereto by the wearer.

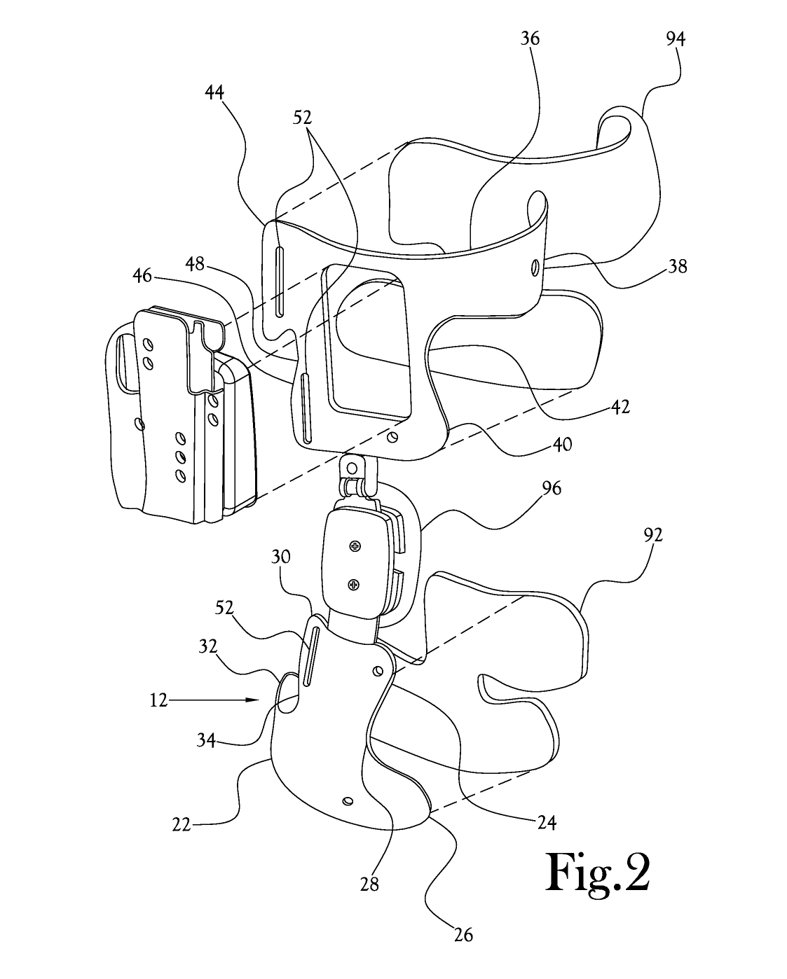

FIG. 2 illustrates a partial exploded view of the knee brace holster 10 of FIG. 1, Referring to FIGS. 1 and 2, in the present embodiment, the lower leg portion 12 includes a first conforming member 22 which is generally sized and shaped to extend along and conform to a portion of a wearer's lower leg, proximate the wearer's calf. In the illustrated embodiment, the first conforming member 22 is defined by a curved plate of substantially rigid material which is generally contoured to an outer calf portion of a wearer's lower leg. In another embodiment, the first conforming member 22 is defined by a panel of flexible material, such as for example fabric, plastic, or the like. In the illustrated embodiment, the first conforming member 22 defines upper and lower front side protuberances 24, 26, respectively, which extend generally outwardly from a front longitudinal side 28 of the first conforming member 22 along the wearer's lower leg and in a direction generally orthogonal to a long dimension of the wearer's lower leg. The first conforming member 22 further defines upper and lower rear side protuberances 30, 32, respectively, which extend generally outwardly from a rear longitudinal side 34 of the first conforming member 22 along the wearer's lower leg and in a direction generally orthogonal to a long dimension of the wearer's lower leg. However, it will be recognized that the first conforming member 22 may define any of a large number of perimetral shapes without departing from the spirit and scope of the present general inventive concept.

The upper leg portion 14 includes a second conforming member 36 which is generally sized and shaped to extend along and conform to a portion of a wearer's upper leg, proximate the wearer's thigh. Similarly to the first conforming member 22 discussed above, the second conforming member 36 is, in the illustrated embodiment, defined by a curved plate of substantially rigid material which is generally contoured to an outer thigh portion of a wearer's lower leg, and in other embodiments, may be defined by a panel of flexible material, such as for example fabric, plastic, or the like. Also similarly to the first conforming member 22 discussed above, the second conforming member 36 defines upper and lower front side protuberances 38, 40, respectively, which extend generally outwardly from a front longitudinal side 42 of the second conforming member 36 along the wearer's upper leg and in a direction generally orthogonal to a long dimension of the wearer's upper leg. The second conforming member 36 further defines upper and lower rear side protuberances 44, 46, respectively, which extend generally outwardly from a rear longitudinal side 48 of the second conforming member 36 along the wearer's upper leg and in a direction generally orthogonal to a long dimension of the wearer's upper leg. As with the first conforming member 22 discussed above, it will be recognized that the second conforming member 36 may define any of a large number of perimetral shapes without departing from the spirit and scope of the present general inventive concept.

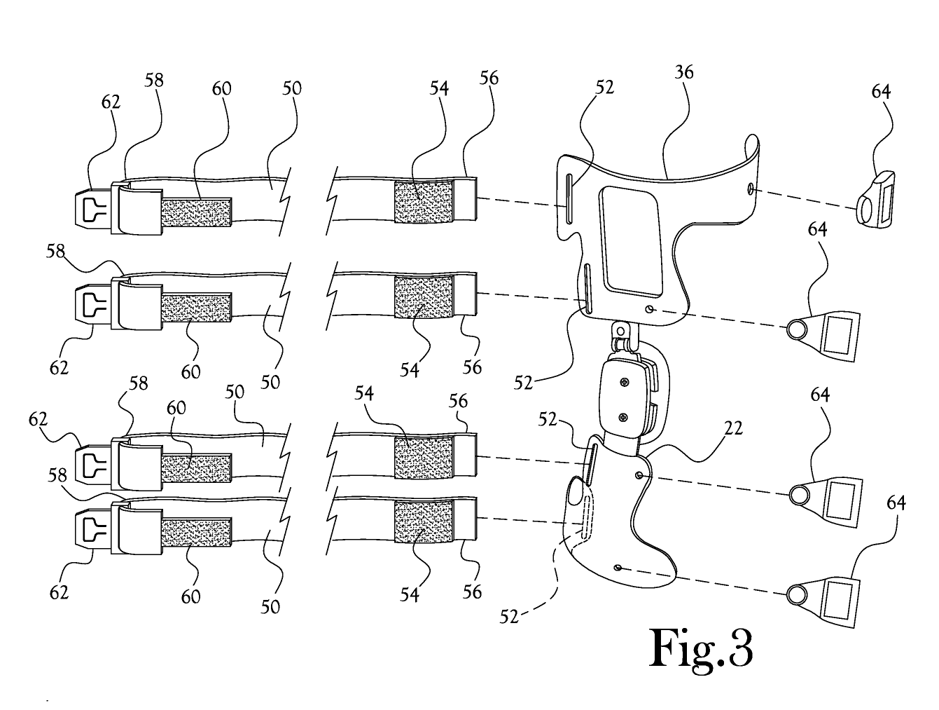

With reference to FIGS. 2 and 3, the knee brace holster 10 further includes a plurality of adjustable wraps 50 configured to secure the first and second conforming members 22, 36 in place against the outer portions of the wearer's lower and upper leg, respectively. In the illustrated embodiment, each of the upper and lower rear side protuberances 30, 32, 44, 46 of the first and second conforming members 22, 36 defines a through slot 52 extending generally parallel to the long dimension of the wearer's leg. Each through slot 52 is sized to receive therethrough one or more flexible wraps 50, which are in turn configured to wrap around the wearer's leg and may be secured to an opposite side of a corresponding conforming member 22, 36 proximate the upper and lower front side protuberances 30, 32, 44, 46 of the conforming member, thereby securing the conforming member 22, 36 along the outer portion of the wearer's leg. In the illustrated embodiment, each wrap 50 is secured to a slot 52 in a respective upper or lower rear side protuberance 30, 32, 44, 46 via a first hook and loop fastener 54 disposed at a first end 56 of the wrap 50. A second end 58 of each wrap 50 defines a second hook and loop fastener 60 which operates to secure a first half 62 of a side release buckle to the wrap second end 58. For each side release buckle first half 62, a corresponding second half 64 of a side release buckle is secured to a corresponding one of the upper or lower front side protuberances 24, 26, 38, 40. Thus, in the illustrated embodiment, upon placing the first conforming member 22 against the outer portion of the wearer's lower leg and the second conforming member 36 against the outer portion of the wearer's upper leg, each wrap 50 may be wrapped around corresponding portions of the wearer's leg. Thereafter, each side release buckle first half 62 may be connectably mated with a corresponding side release buckle second half 64, thereby securing each conforming member 22, 36 along its respective outer portion of the wearer's leg.

In several embodiments, the effective length of each wrap 50 is selectively adjustable. For example, in the illustrated embodiment, the various hook and loop fasteners 54, 60 disposed along first and second ends 56, 58 of the wraps 50 allow for selective adjustability of the positioning of each side release buckle first half 62 along the length of a corresponding wrap 50, as well as the positioning of each slot 52 along a the length of a corresponding wrap 50. Thus, the hook and loop fasteners 54, 60 allow for selective adjustability of the effective length of each wrap 50. However, it will further be recognized that other suitable configurations and devices may be employed to allow fastening of the wraps 50 to the first and second conforming members 22, 36, and to accomplish adjustability of the effective length of the wraps 50, and such other configurations and devices may be used without departing from the spirit and scope of the present general inventive concept. For example, in several embodiments, each wrap 50 is received within a suitable series of slots defined along a corresponding conforming member 22, 36 or along a corresponding side release buckle which allow the effective length of each wrap 50 to be adjusted. In other embodiments, hook and loop fastener components are provided to allow both ends of each wrap 50 to be secured directly to a corresponding conforming member 22, 36.

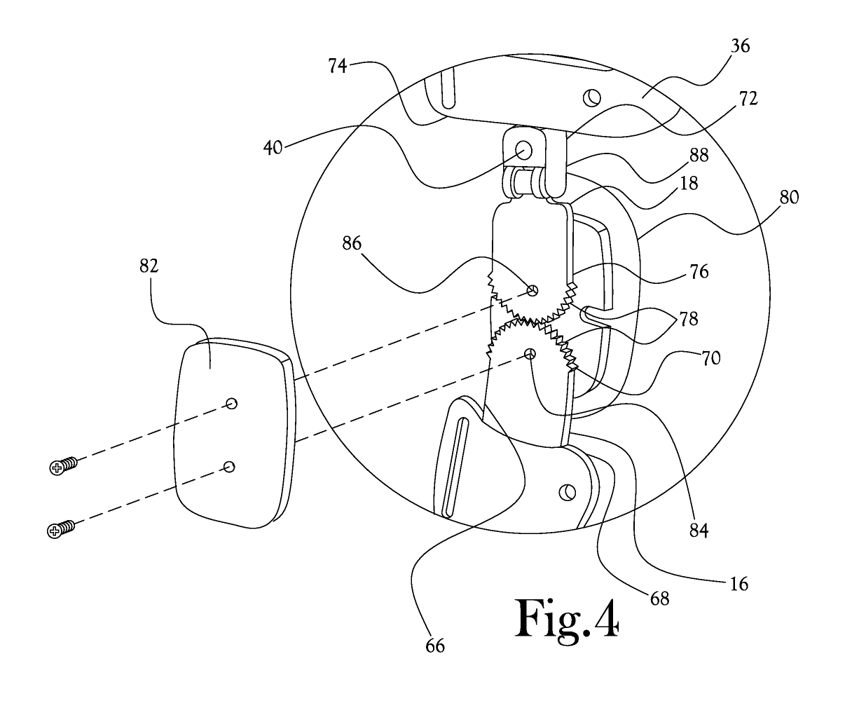

Referring to FIGS. 1, 2, and 4, the lower leg portion 12 is movably, and in several embodiments rotatably, connected to the upper leg portion 14. For example, in the illustrated embodiment, the lower leg portion 12 further includes a first connecting member 16 which is secured at a first end 66 thereof to an upper end 68 of the first conforming member 22. A second end 70 of the first connecting member 16 extends generally upwardly toward the upper leg portion 14. Similarly, the upper leg portion 14 includes a second connecting member 18 which is secured at a first end 72 thereof to a lower end 74 of the second conforming member 36. A second end 76 of the second connecting member 18 extends generally downwardly toward the lower leg portion 12 to establish a rotatable connection with the second end 70 of the first connecting member 16. For example, in the illustrated embodiment, the respective second ends 70, 76 of the first and second connecting members 16, 18 each define rounded profiles with mating, complimentary gear teeth 78 extending radially outwardly therefrom. The second ends 70, 76 of the first and second connecting members 16, 18 are arranged in an end-to-end configuration with the gear teeth of the first connecting member 16 in mating engagement with the gear teeth of the second connecting member 18. Thus, a geared, rotatable connection is formed between the second ends 70, 76 of the first and second connecting members 16, 18. In the illustrated embodiment, inner and outer plates 80, 82 are provided along opposite sides of the mating connection of the first and second connecting members 16, 18. Suitable rotatable fasteners 84, 86 are provided between the inner and outer plates 80, 82 through rotational axes of the first and second connecting members 16, 18 to maintain mating, rotatable engagement of the first and second connecting members 16, 18. Thus, rotation of one of the first and second connecting members 16, 18 about a respective rotatable fastener 84, 86 in relation to the inner and outer plates 80, 82 results in equal and opposite rotation of the other of the first and second connecting members 16, 18 about a respective rotatable fastener 84, 86 in relation to the inner and outer plates 80, 82.

In the illustrated embodiment, the first end 72 of the second connecting member 18 is secured to the second conforming member 36 via a hinge connection 88. The hinge connection 88 is rotatable about an axis which extends generally perpendicular to a rotational axis of the geared, rotatable connection of the second ends 70, 76 of the first and second connecting members 16, 18. Accordingly, in the illustrated embodiment, the lower leg portion 12 and upper leg portion 14 are capable of multi-directional movement in relation to one another. In the illustrated embodiment, a set screw 90 is provided which may be loosened to allow rotational movement of the second connecting member 18 about the upper leg portion 14, and which may be tightened to limit rotational movement of the second connecting member 18 about the upper leg portion 14.

Those of skill in the art will recognize numerous other types of connections which are suitable for use in establishing a movable connection between the lower leg portion 12 and the upper leg portion 14. For example, in one embodiment, the first and second connecting members 16, 18 are connected to one another using a single rotatable pin connection. In another embodiment, the first and second connecting members 16, 18 are connected to one another by a ball and socket joint. In other embodiments, the first and second conforming members 22, 36 are secured to one another via a segment of flexible material, such as for example fabric, rubber, polymer, etc. Accordingly, it will be recognized that numerous other types of connections may be used to establish movable connection between the lower leg portion 12 and the upper leg portion 14 without departing from the spirit and scope of the present general inventive concept.

Referring to FIGS. 1 and 2, in several embodiments, at least one padded lining is provided along an interior of the knee brace holster 10 to improve comfort of the knee brace holster 10 to the wearer. For example, in the illustrated embodiment, the knee brace holster 10 further includes a plurality of substantially flexible pads 92, 94, 96 secured along inner surfaces of the first conforming member 22, second conforming member 36, and the inner plate 80, such that the pads are interposed between the wearer's leg and each of the first conforming member 22, the second conforming member 36, and the first and second connecting members 16, 18 when the knee brace holster 10 is worn. Each pad 92, 94, 96 is sized and shaped to conform generally along its corresponding inner surface and also to conform along associated portions of the wearer's leg. In the illustrated embodiment, a first pad 92 is provided along an inner surface of the first conforming member 22. The first pad 92 defines a perimetral shape which is similar to, but slightly larger than, the perimetral shape of the first conforming member 22, such that the various edges of the first pad 92 slightly overhang respective edges of the first conforming member 22. Likewise, a second pad 94 is provided along an inner surface of the second conforming member 36 which has a perimetral shape similar to, but slightly larger than, the perimetral shape of the second conforming member 36, such that the various edges of the second pad 94 slightly overhang respective edges of the second conforming member 36. A third pad 96 is provided along an inner surface of the inner plate 80 of the interface between the first and second connecting members 16, 18, and has a perimetral shape similar to, but slightly larger than, the perimetral shape of the inner plate 80. Thus, the various edges of the third pad 96 slightly overhang respective edges of the inner plate 80.

In certain embodiments, each pad 92, 94, 96 is fixed along its associated inner surface 92, 94, 96 as by adhesive or other suitable fastener. In other embodiments, each pad 92, 94, 96 is removably secured along its associated inner surface 92, 94, 96 as by suitable releasable fasteners, such as for example hook and loop fasteners, frictional connections, snaps, or the like. It will be understood that other fasteners exist which are suitable for securing the pads 92, 94, 96 along the inner surfaces 92, 94, 96. It will be recognized that the pads 92, 94, 96 may define any of a large number of perimetral shapes without departing from the spirit and scope of the present general inventive concept. Furthermore, it will be understood that inclusion of the pads 92, 94, 96 is not necessary to accomplish the present general inventive concept.

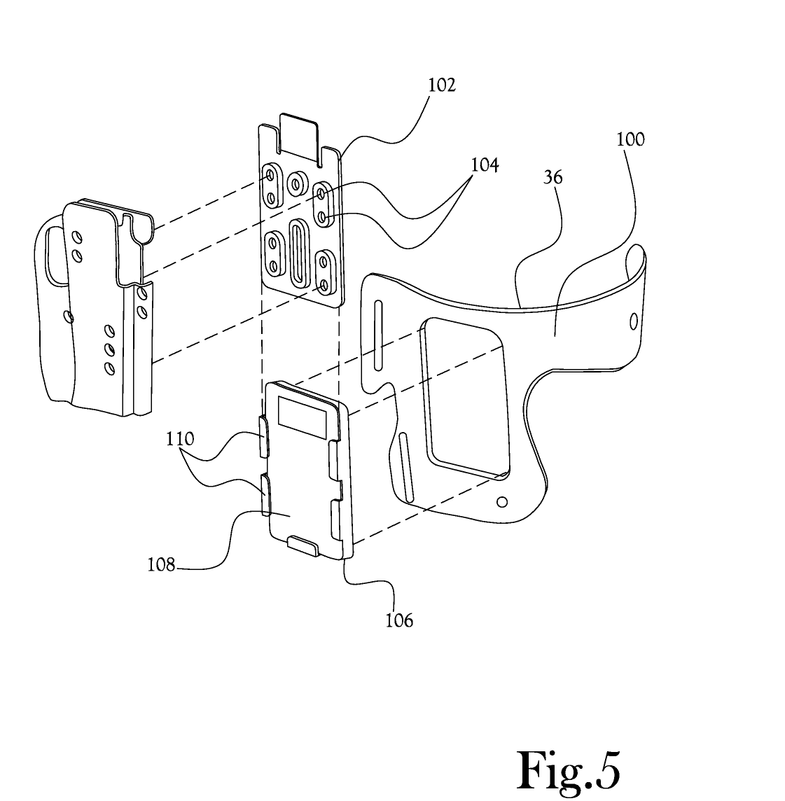

As discussed above, at least one exterior surface of the leg portions 12, 14 of the knee brace holster 10 is adapted to carry a holster 20 secured thereto. In some embodiments, the holster is fixed against a corresponding outer surface of the leg portions 12, 14. In other embodiments, the holster 20 is removably secured against a respective outer surface 98, 100 of the leg portions 12, 14. For example, in the embodiment of FIGS. 1 and 5, a mounting bracket 102 is provided which is adapted to be secured to the outer surface 100 of the upper leg portion 14. The mounting bracket 102 is also sized and shaped to allow mounting of a holster 20 thereto. In several embodiments, the mounting bracket 102 defines suitable fasteners to assist in fastening the mounting bracket 102 to at least one holster 20. For example, in the illustrated embodiment the mounting bracket 102 defines a plurality of through bores 104 adapted to mate with and receive a fastening mechanism of the holster 20, such as for example a screw, pin, tab, or other such fastening mechanism. In another embodiment, the mounting bracket 102 is fixed to the holster by an integral connection. Those skilled in the art will recognize other suitable devices for securing the mounting bracket 102 to the holster 20, and such devices may be used without departing from the spirit and scope of the present general inventive concept.

In some embodiments, the mounting bracket 102 is directly securable to the at least one outer surface 98, 100 of the leg portions 12, 14. In other embodiments, such as the embodiment of FIGS. 1-6, the mounting bracket 56 is securable to a mounting sleeve 106, which is in turn secured to one of the outer surfaces 98, 100. In several embodiments, the mounting sleeve 106 may be secured to an outer surface 98, 100, such as for example by a suitable fastener or by integral connection. The mounting sleeve 106 includes an outer surface 108 defining a plurality of lips 110 overhanging the outer surface 108 along peripheral edges of the outer surface 108. The lips 110 are configured to allow slidable receipt of the mounting bracket 102 between the lips 110 and an outer surface of the mounting sleeve 106, and to limit separation of the received portion of the mounting bracket 102 from the outer surface 110 in a direction substantially perpendicular to the outer surface 110. Thus, with the portion of the mounting bracket 102 slidably received between the lips 110 and the mounting block outer surface 108, one or more fastening mechanisms may be configured to engage the mounting bracket 102 to prevent slidable withdrawal of the holster 20 and associated mounting bracket 102 from the mounting sleeve 106.

It will be understood that, in addition to the illustrated embodiments, the mounting bracket 102 may be sized and shaped in any of a large number of configurations to allow mounting of a holster 20 to an outer surface 98, 100 of the leg portions 12, 14 through conventional means of the type known to one of ordinary skill in the art. Furthermore, it will be understood that the knee brace holster 10 may include any of a large number of shapes and configurations of holsters 20 for securing numerous types of articles 112 to a wearer's leg without departing from the spirit and scope of the present invention. For example, in the illustrated embodiment, the knee brace holster 10 includes a holster 20 of the type suitable for receiving and carrying a firearm 112 secured to an outer surface 98, 100 of the leg portions 12, 14. More specifically, the holster 20 as shown in FIG. 1 is a polymer-type retention holster for a firearm. In the embodiment of FIG. 6, the knee brace holster 10′ includes a first holster 20′ which is adapted to hold and carry a firearm magazine 112′ and a second holster 20″ which is adapted to hold and carry a pair of handcuffs 112″.

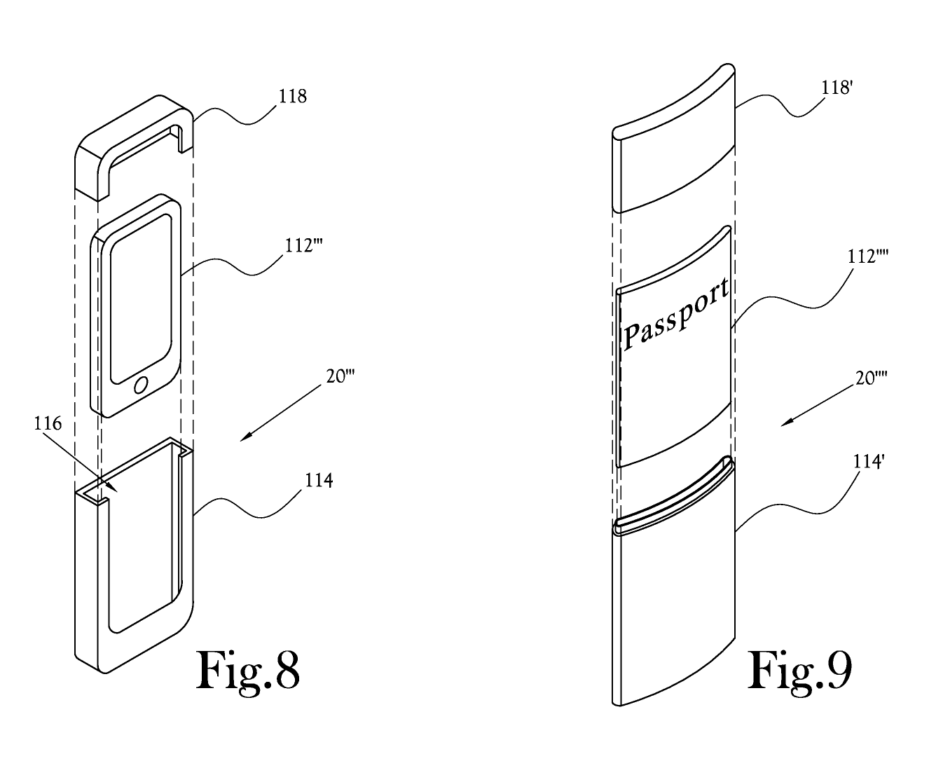

FIG. 8 illustrates another type of holster 20′″ which may be used with the knee brace holster 10 to hold and carry a cellular device 112′″, such as for example a cellular phone, personal digital assistant, tablet, electronic reader, or other such device. As shown in FIG. 8, in one embodiment, the holster 20′″ includes a lower housing 114 defining an interior cavity 116 which is sized and shaped to receive a cellular device 112′″ therein. The holster 20′″ may optionally include a cap 118 which is configured to at least partially seal the interior cavity 116 to limit removal of the cellular device 112′″ therefrom. In the embodiment of FIG. 9, the holster 20″″ is configured to hold and carry personal documents 112″″, such as for example a passport, identification, or other personal documents. In the embodiment of FIG. 9, the lower housing 114′ and cap 94′ are contoured to better conform to the leg of a wearer during use. In addition to the above-discussed embodiments, it will be recognized by one of skill in the art that the holster 20 may be a configured to hold any of several numbers and types of articles, such as for example firearms, ammunition, ammunition magazines, flashlights, knives, hand tools, navigation devices, communication devices, aiming devices, personal protection devices, non-lethal weapons, handcuffs and other restraint devices, personal documents, cards and identification, or the like, without departing from the spirit and scope of the present general inventive concept.

From the foregoing description, it will be understood that a knee brace holster 10 has been described herein which provides a fastening means for securing a holster proximate a leg of a wearer with improved stability of the holster in relation to the wearer's leg. Furthermore, it will be understood that the knee brace holster of the present general inventive concept provides improved support to help stabilize the associated knee of the wearer, while also allowing for improved comfort to the wearer. It will be understood that, when a knee brace holster constructed in accordance with the present general inventive concept is worn by a wearer, the knee brace holster remains snugly in place along the wearer's leg throughout movement of the leg, thereby improving the stability of the holster in relation to the wearer's leg, discouraging injury to the wearer, and allowing for increased ease of use of the holster.

While the present invention has been illustrated by description of several embodiments and while the illustrative embodiments have been described in detail, it is not the intention of the applicant to restrict or in any way limit the scope of the appended claims to such detail. Additional modifications will readily appear to those skilled in the art. The invention in its broader aspects is therefore not limited to the specific details, representative apparatus and methods, and illustrative examples shown and described. Accordingly, departures may be made from such details without departing from the spirit or scope of applicant's general inventive concept.

Having thus described the aforementioned invention, what is claimed is:

1. An knee brace holster comprising:
a knee brace configured to wrap around at least a portion of a leg of a user proximate a knee of the user to provide support to the user's knee; and
a mount for mounting a holster to an outer surface of said knee brace.

2. The knee brace holster of claim 1, said knee brace further comprising:
an upper portion configured to wrap around at least a portion of the user's leg above the user's knee; and
a lower portion configured to wrap around at least a portion of the user's leg below the user's knee.

3. The knee brace holster of claim 2, said upper portion being rotatably connected to said lower portion.

4. The knee brace holster of claim 2, said knee brace further including a pair of padded linings, each padded lining being disposed along and being sized and shaped to conform to an inner surface of one of said upper and lower portions.

5. The knee brace holster of claim 4, each said padded lining being releasably secured to a corresponding inner surface of one of said lateral and medial plates.

6. The knee brace holster of claim 2, said lower portion further comprising:
a first substantially rigid plate shaped to conform to at least a portion of the user's leg below the user's knee; and
at least a first flexible wrap secured at a first end thereof to a first side of said first rigid plate and securable at a second end thereof to a second side of said first rigid plate.

7. The knee brace holster of claim 6, said upper portion further comprising:
a second substantially rigid plate shaped to conform to at least a portion of the user's leg above the user's knee; and
at least a second flexible wrap secured at a first end thereof to a first side of said second rigid plate and securable at a second end thereof to a second side of said second rigid plate.

8. The knee brace holster of claim 7, said first flexible wrap being securable to said second side of said first rigid plate via a first side release buckle, and said second flexible wrap being securable to said second side of said second rigid plate via a second side release buckle.

9. The knee brace holster of claim 1, said mount including at least one mounting bracket securable to said knee brace, said mounting bracket being shaped to allow a holster to be mounted thereto.

10. The knee brace holster of claim 8, said mount further including at least one mounting sleeve secured to said knee brace, said mounting sleeve being sized and shaped to engage said mounting bracket to secure said mounting bracket to said mounting sleeve.

11. The knee brace holster of claim 10 further including a holster mounted to said mounting bracket.

12. The knee brace holster of claim 11, said holster being fabricated from a material selected from the group consisting of polymer, leather, and fabric.

13. The knee brace holster of claim 12, said holster being shaped to receive therein at least one of a firearm, a unit of ammunition, an ammunition magazine, a flashlight, a knife, a hand tool, a navigation device, a communication device, an aiming device, a personal protection device, a non-lethal weapon, and a pair of handcuffs.

14. The knee brace holster of claim 13, said holster being a retention holster.

15. The knee brace holster of claim 14, said knee brace further including at least one padded lining disposed along an interior surface of said knee brace.
